(19) United States
(12) Patent Application Publication
(10) Pub. No.: US 2012/0183927 A1
(43) Pub. Date: Jul. 19, 2012

(54) Dental Compositions for Improving Brushing Habits

(76) Inventor: Ian I. Cho, Tucson, AZ (US)

(21) Appl. No.: 13/335,464

(22) Filed: Dec. 22, 2011

Related U.S. Application Data
(60) Provisional application No. 61/432,385, filed on Jan. 13, 2011.

(51) Int. Cl. A61C 17/00; A61K 49/00

(52) U.S. Cl. 433/216; 424/9.7

(57) ABSTRACT

Dental compositions including a dye, and in some embodiments dental plaque-disclosing dye, and an adhesive agent. Kits and methods involving the compositions help users to determine proper brushing habits in terms of time, pressure, and brush used based on the removal from tooth enamel of the dye and adhesive mixture in the compositions.

### CROSS REFERENCE TO RELATED APPLICATIONS

This application claims priority from U.S. Patent Application Ser. No. 61/432,385 filed Jan. 13, 2011.

### FIELD OF INVENTION

The subject matter herein concerns dental compositions, and, more particularly, improved compositions including a dye or brushing indicator for giving feedback on the thoroughness of brushing for a user.

### DESCRIPTION OF THE RELATED ART

When brushing one's teeth, it is important to brush with sufficient technique (which may include strength, pattern, and location of brushing strokes) and equipment such that plaque is effectively removed. Yet, one also must be careful not to brush so hard, with very stiff bristles, or too long such that the enamel of the teeth is damaged.

Thus, while attempts have been made to give feedback about the level of plaque that remains post brushing (e.g., through “disclosure tablets”), such compositions are not known to effectively give feedback during the brushing process such that a user can observe the effectiveness of plaque removal while helping to ensure that brushing duration and other parameters do not exceed what is needed for plaque removal, thereby helping to minimize damage to tooth enamel.

### SUMMARY OF INVENTION

The subject matter generally relates to compositions, kits, and methods that include a dental plaque-disclosing dye or other dye and an adhesive agent (such as a glue, paste, mastic, or other sticky substance) that adheres to tooth enamel such that the adhesive agent and dye are removed through the removal of dental plaque.

It is envisioned that the subject matter of this application will be especially useful in teaching good tooth-brushing habits to children. Most children do not intentionally refuse to brush their teeth; instead, they are in a hurry, do not think about it, or have not had teeth brushing instilled as a natural part of their routine.

Hence, the compositions include a dye that coats teeth. The dye is designed to be removed through thorough tooth brushing. Consequently, one positive-habit effect is that teeth are colored and a “game” is possible for teaching children good teeth brushing habits as tooth brushing to remove food debris and dental plaque is required to substantially remove the composition.

In one embodiment, the dental compositions solely include a dental plaque-disclosing dye and an adhesive agent that adheres the dye to tooth enamel such that plaque removal removes the dye.

In another embodiment, the dental compositions further include xylitol, sodium fluoride, ethanol, glycerin, silicon dioxide, and/or a polyethylene glycol, such as PEG-8.

In another embodiment, the dental compositions include a dye and an agent that helps the dye to adhere to tooth enamel such that brushing with good equipment and technique for a sufficient time removes the dye.

The subject matter herein also involves methods for obtaining real-time feedback on tooth brushing efficacy that include applying a dental composition to the teeth, the composition including a dental plaque-disclosing dye and/or other dye and an adhesive agent, and then brushing teeth such and until the dye is substantially reduced or removed from tooth enamel.

To minimize or avoid damage to tooth enamel, the methods also involve ceasing brushing at such time as the dye is substantially reduced or removed from the teeth of the brusher.

Various other purposes and advantages of the invention will become clear from its description in the specification that follows. Therefore, to the accomplishment of the objectives described above, this invention includes the features hereinafter fully described in the detailed description of the preferred embodiments, and particularly pointed out in the claims. However, such description discloses only some of the various ways in which the invention may be practiced.

### DETAILED DESCRIPTION OF THE PREFERRED EMBODIMENTS

The subject matter herein relates to a dental composition, comprising at least one dye and an adhesive agent, wherein the adhesive agent adheres the dye to tooth enamel such that brushing is required to remove or reduce the dye.

The subject matter herein relates in one embodiment to a colored varnish form of dentifrice which will coat and stick to teeth upon contact with saliva. For example, the dentifrice may be a composition including by weight:

Natural rosins—varnish matrix 50-60%

Ethanol—carrier 10-20%

Glycerin—humectant 5-10%

Silicon dioxide—abrasive 5-10%

PEG-8 (polyethylene glycol 8)—dispersant 2-4%

Xylitol—sweetener 1-2%

FDC Red #40—coloring 0.5-1%

FDC Blue #1—coloring 0.5-1% and optionally sodium fluoride (0.15% NO; fluoride ion)—anti-cavity

The ingredients are mixed in order until blended into a varnish suitable for application to the teeth with, for example, an applicator brush.

Normally it takes 2 minutes to 3 minutes of thorough brushing with a soft bristle brush applied with light-to-medium pressure to remove the dye in the composition above. Since this is the period that most professionals suggest you brush your teeth, an embodiment such as that above helps to ensure thorough brushing and plaque removal while providing enamel enhancing fluoride.

In an embodiment of the compositions, the sole or at least one dye may be a dental plaque-disclosing dye, such as a solution of FDC Red No. 3 (200 mg), FDC Green No. 3 (400 mg), surfactant (Tureen 20; 0.1 ml), and tap water (60 ml).

In a kit embodiment, a dental composition, comprising at least one dye and an adhesive agent, and a toothbrush is provided. The adhesive agent may be a glue, paste, mastic, or other sticky substance that adheres the dye to tooth enamel such that normal brushing (i.e., light-to-medium pressure with a soft-bristle manual brush) for about 2-3 minutes is required to remove or reduce the dye. Resins, for example, as are used in fluoride varnishes, may be the adhesive.

Methods for obtaining real-time feedback of tooth-brushing efficacy also are provided. In one embodiment, the method involves performing the steps of: (1) applying a dental composition to the teeth, the composition including a dental plaque-disclosing dye and/or other dye and an adhesive agent, and (2) brushing teeth such and until the dye is substantially reduced (i.e., until a light shade of the original color remains) or removed from tooth enamel.

Advantageously, compositions, kits and methods of the subject matter can provide feedback on whether tooth brushing may be damaging to tooth enamel because, for example, the dye is removed very quickly (a possible indication of brushing too hard or with very hard bristles) or when typical brushing was extending beyond the duration needed for effective plaque removal.

Various changes in the details and components that have been described may be made by those skilled in the art within the principles and scope of the invention herein described in the specification and defined in the appended claims. Therefore, while the present invention has been shown and described herein in what is believed to be the most practical and preferred embodiments, it is recognized that departures can be made there from within the scope of the invention, which is not to be limited to the details disclosed herein but is to be accorded the full scope of the claims so as to embrace any and all equivalent processes and products. All references cited in this application are hereby incorporated by reference herein.

What is claimed is:

1. A dental composition, comprising at least one dye mixed with an adhesive agent, wherein the adhesive agent adheres the dye to tooth enamel such that normal brushing for about 2-3 minutes is required to remove or substantially reduce the dye.

2. The composition of claim 1, wherein the at least one dye is a dental plaque-disclosing dye.

3. The composition of claim 1, further including a xylitol, sodium fluoride, ethanol, glycerin, silicon dioxide, and a polyethylene glycol.

4. The composition of claim 1, wherein the dental composition is a varnish that includes by weight:
50%-60% of a natural rosin as the varnish matrix.
10%-20% ethanol,
5%-10% glycerin,
5%-10% silicon dioxide,
2%-4% polyethylene glycol 8,
1%-2% xylitol,
0.5%-1% FDC Red #40, and
0.5%-1% FDC Blue #1.

5. A kit comprising a dental composition containing at least one dye and an adhesive agent, wherein the adhesive agent adheres the dye to tooth enamel such that normal brushing for about 2-3 minutes is required to remove or substantially reduce the dye, and a toothbrush.

6. The kit of claim 5, wherein the dental composition containing at least one dye and an adhesive agent is a varnish that includes by weight:
50%-60% of a natural rosin as the varnish matrix,
10%-20% ethanol,
5%-10% glycerin,
5%-10% silicon dioxide,
2%-4% polyethylene glycol 8,
1%-2% xylitol,
0.5%-1% FDC Red #40, and
0.5%-1% AFDC Blue #1.

7. In a method for obtaining real-time feedback of tooth-brushing efficacy, performing the steps of: (1) applying a dental composition to the teeth, the composition including at least one dye mixed with an adhesive agent, wherein the adhesive agent adheres the dye to tooth enamel such that normal brushing for about 2-3 minutes is required to remove or substantially reduce the dye, and (2) brushing said tooth enamel until the dye is substantially reduced or removed therefrom.

8. The method of claim 7, wherein the dental composition is a varnish that includes by weight:
50%-60% of a natural rosin as the varnish matrix,
10%-20% ethanol,
5%-10% glycerin,
5%-10% silicon dioxide,
2%-4% polyethylene glycol 8,
1%-2% xylitol,
0.5%-1% FDC Red #40, and
0.5%-1% AFDC Blue #1.
